(19) United States
(12) Patent Application Publication
(10) Pub. No.: US 2004/0249401 A1
(43) Pub. Date: Dec. 9, 2004

(54) Apparatus and method for an ultrasonic medical device with a non-compliant balloon

(75) Inventors: Robert A. Rabiner, North Reading, MA (US); Bradley A. Hare, Chelmsford, MA (US)

Correspondence Address: PALMER & DODGE, LLP, RICHARD B. SMITH, 111 HUNTINGTON AVENUE, BOSTON, MA, 02199, US

(73) Assignee: OmniSonics Medical Technologies, Inc.

(21) Appl. No.: 10/846,037

(22) Filed: May 14, 2004

Related U.S. Application Data
(63) Continuation-in-part of application No. 10/665,445, filed on Sep. 19, 2003.
Application No. 10/665,445 is a continuation of application No. 09/776,015, filed on Feb. 2, 2001, now Pat. No. 6,652,547.
Application No. 09/776,015 is a continuation-in-part of application No. 09/618,352, filed on Jul. 19, 2000, now Pat. No. 6,551,337.
(60) Provisional application No. 60/178,901, filed on Jan. 28, 2000.
(60) Provisional application No. 60/157,824, filed on Oct. 5, 1999.

(51) Int. Cl. A61B 17/22

(52) U.S. Cl. 606/159

(57) ABSTRACT

The present invention provides an apparatus and a method for an ultrasonic medical device with a non-compliant balloon used to ablate a biological material. The ultrasonic medical device comprises an ultrasonic probe, a catheter surrounding the ultrasonic probe, a balloon supported by the catheter and an inflation lumen located along a longitudinal axis of the catheter. The ultrasonic probe is inserted into a vasculature and the catheter comprising the balloon is advanced until the balloon is adjacent to the biological material. The balloon is inflated until at least a portion of an outer surface of the balloon engages the biological material. The ultrasonic probe is energized to produce a transverse ultrasonic vibration that vibrates at least a portion of the balloon to ablate the biological material into a particulate.

### RELATED APPLICATIONS

This application is a continuation-in-part of application Ser. No. 10/665,445, filed Sep. 19, 2003, which is a continuation of application Ser. No. 09/776,015, filed Feb. 2, 2001, now U.S. Pat. No. 6,652,547, which is a continuation-in-part of application Ser. No. 09/618,352, filed Jul. 19, 2000, now U.S. Pat. No. 6,551,337, which claims benefit of Provisional Application Ser. No. 60/178,901, filed Jan. 28, 2000, and claims benefit of Provisional Application Ser. No. 60/157,824, filed Oct. 5, 1999, the entirety of all these applications are hereby incorporated herein by reference.

### FIELD OF THE INVENTION

The present invention relates to an ultrasonic medical device, and more particularly to an apparatus and method for an ultrasonic medical device with a non-compliant balloon used to ablate a biological material.

### BACKGROUND OF THE INVENTION

Vascular occlusive disease affects millions of individuals worldwide and is characterized by a dangerous blockage of vasculatures. Vascular occlusive disease includes thrombosed hemodialysis grafts, peripheral artery disease, deep vein thrombosis, coronary artery disease, heart attack and stroke. Vasculatures include veins, arteries, blood vessels, intestines, ducts and other body lumens that materials may flow through. Vascular occlusions (clots, intravascular blood clots or thrombus, occlusional deposits, such as calcium deposits, fatty deposits, atherosclerotic plaque, cholesterol buildup, fibrous material buildup, arterial stenoses) result in the restriction or blockage of blood flow in the vessels in which they occur. Adequate functioning of various organs in the body relies on the delivery of nutrients and oxygen via blood. Occlusions result in oxygen deprivation (“ischemia”) of tissues supplied by these blood vessels. Prolonged ischemia results in permanent damage of tissues which can lead to myocardial infarction, stroke, or death. Targets for occlusion include coronary arteries, peripheral arteries and other blood vessels.

The disruption of an occlusion can be affected by pharmacological agents, mechanical methods, ultrasonic methods or combinations of all three. Many thrombolytic drugs are associated with side effects such as severe bleeding which can result in a cerebral hemorrhage. Mechanical methods of treating an occlusion or a thrombus result in large forces applied to the vessel which can result in vessel rupture or weakening of the vessel. Weakening of the vessel increases the probability of post-operative aneurysm of the creation of vasoconstrictive or restenotic conditions.

Balloon angioplasty, a mechanical method of treating an occlusion, is generally limited to use in large blood vessels. Balloon angioplasty, also referred to as angioplasty or PTCA (percutaneous transluminal coronary angioplasty), is a minimally invasive, non-surgical method of treating an occlusion to remove the occlusion and open the vasculature to allow blood to circulate. Several methods of balloon angioplasty are known in the art. For example, a catheter is inserted into the vasculature of the body and an x-ray of the vasculature is taken to measure the extent of the narrowing of the vasculature. After the blockage is located, a guidewire is advanced to the site of the occlusion and a second catheter with a balloon located on it is passed over the guidewire. The second catheter is advanced to the occlusion and the balloon is inflated. The inflation of the balloon presses the occlusion against the walls of the vasculature and the balloon is subsequently deflated.

As a result of the large forces applied to the vessel walls, the vessel walls may scar and lead to the formation of a secondary occlusion, a process known as restenosis. Abrupt closure of the vessels or spasms of the vessels, a process known as vasoconstriction, may also occur. These post procedure conditions require an additional procedure including, but not limited to, angioplasty, a bypass operation and/or stenting. Additional or repeat procedures are undesirable since the patient is subjected to an additional procedure which carries a higher risk of complications. Additional forces from subsequent procedures put additional stresses on the vessels which may weaken the vessels.

Restenosis is a condition in which the vasculature re-narrows or blocks completely after a previous treatment method such as an angioplasty or stenting treatment. Restenosis is a common problem, with about one third to about one half of patients returning for a subsequent treatment within about three to about six months after a previous treatment procedure. In restenosis, there is an overproduction of cells, similar to scar tissue, at the site where the previous treatment procedure was carried out. The overproduction of cells at the site of the occlusion results in the diameter of the vasculature at the occlusion site reducing in size and ultimately closing.

A vasoconstriction is a sudden decrease in the diameter of the vasculature resulting from the contraction of smooth muscle within the wall of the vasculature. The diameter reduction leads to a decrease in blood flow and to a condition known as an increase in systematic vascular resistance. Systematic vascular resistance results in the vasculature undergoing a series of spasms.

In order to prevent restenosis and vasoconstriction, stenting procedures may be performed. Stenting is a catheter based procedure in which a stent is inserted into a vasculature of a body. A stent is a tube made of metal wire or plastic that is inserted into the vasculature of the body to keep the vasculature open and prevent closure of the vasculature. A stent is a permanent device that becomes a part of the cardiovascular system. Often, stenting is performed in conjunction with other catheter based procedures including, but not limited to, balloon angioplasty and atherectomy. In a typical stenting procedure, a guide catheter is advanced through a sheath to a site of the occlusion. A stent with a balloon-tipped catheter inside the walls of the stent is advanced to the site of the occlusion and the balloon is inflated to expand the stent. The expansion of the stent allows the stent to engage to the wall of the vasculature. The balloon catheter is removed while the stent remains engaged to the walls of the vasculature.

The use of a balloon to open up the vasculature for blood flow is discussed in the prior art (see, e.g., U.S. Pat. No. 6,491,711 and U.S. Pat. No. 6,503,223). Prior art use of a balloon compresses the occlusive material against the walls and does not completely remove the occlusive material from the vasculature of the body. Additional techniques of utilizing two balloons to isolate a chamber between the balloons is discussed in the prior art (see, e.g., U.S. Pat. No. 5,833,650; U.S. Pat. No. 5,919,163 and U.S. Pat. No. 6,475,185). Prior art patents utilizing two balloons are not efficient at breaking up the occlusive material and do not solve the problem of subsequent restenosis and vasoconstriction.

The use of ultrasonic probes to treat body tissue have been used in many surgical procedures (see, e.g., U.S. Pat. No. 5,112,300; U.S. Pat. No. 5,180,363; U.S. Pat. No. 4,989,583; U.S. Pat. No. 4,931,047; U.S. Pat. No. 4,922,902; and U.S. Pat. No. 3,805,787). The use of ultrasonic energy has been proposed both to mechanically disrupt clots, and to enhance the intravascular delivery of drugs to clot formations (see, e.g., U.S. Pat. No. 5,725,494; U.S. Pat. No. 5,728,062; and U.S. Pat. No. 5,735,811). Ultrasonic devices used for vascular treatments typically comprise an extracorporeal transducer coupled to a solid metal wire which is then threaded through the blood vessel and placed in contact with the occlusion (see, e.g., U.S. Pat. No. 5,269,297). In some cases, the transducer, comprising a bendable plate, is delivered to the site of the clot (see, e.g., U.S. Pat. No. 5,931,805).

Some ultrasonic devices include a mechanism for irrigating an area where the ultrasonic treatment is being performed (e.g., a body cavity or lumen) in order to wash tissue debris from the area of treatment. Mechanisms used for irrigation or aspiration described in the prior art are generally structured such that they increase the overall cross-sectional profile of the elongated probe, by including inner and outer concentric lumens within the probe to provide irrigation and aspiration channels. In addition to making the probe more invasive, prior art probes also maintain a strict orientation of the aspiration and the irrigation mechanism, such that the inner and outer lumens for irrigation and aspiration remain in a fixed position relative to one another, which is generally closely adjacent to the area of treatment. Thus, the irrigation lumen does not extend beyond the suction lumen (i.e., there is no movement of the lumens relative to one another) and any aspiration is limited to picking up fluid and/or tissue remnants within the defined area between the two lumens.

As discussed above, medical devices utilizing ultrasonic energy to destroy occlusive material in the human body are known in the art. A major drawback of existing ultrasonic devices having a probe for occlusive material removal is that they are relatively slow in comparison to procedures that involve surgical excision. This is mainly attributed to the fact that such ultrasonic devices rely on imparting ultrasonic energy to contacting occlusive material by undergoing a longitudinal vibration of the probe tip, wherein the probe tip is mechanically vibrated at an ultrasonic frequency in a direction parallel to the probe longitudinal axis. This, in turn, produces an occlusive material destroying effect that is entirely localized at the probe tip, which substantially limits its ability to remove large occlusive material areas in a short time. An ultrasonic medical device with a multiple material coaxial construction for conducting axial vibrations is known in the art (see, e.g., U.S. Pat. No. 6,277,084).

In addition to prior art ultrasonic devices being slow and not focusing energy at the sites of the occlusion, previous ultrasonic methods of treating plaque still include many undesirable complications and dangers. The ultrasonic energy can adversely affect areas of healthy tissue within the vasculatures of the body that are in proximity to the occluded areas. There is no dampening medium that can better protect the areas of healthy tissue, while helping to focus the ultrasonic energy at the sites of the occlusion.

Prior art attempts at removing an occlusion without damaging the vasculature or requiring the need for a subsequent procedure have been less than successful. Some prior art devices use a balloon and heat to soften the occlusion and increase the risk of further health complications (see e.g., U.S. Pat. No. 4,924,863; U.S. Pat. No. 6,123,718; U.S. Pat. No. 5,057,106 and U.S. Pat. No. 5,575,772). These prior art devices are bulky and require a complicated system of wires and heating components used for the treatment of the occlusive material. Some of these prior art devices require a cooling system to reduce the risk of further health complications of the individual undergoing the procedure. Other prior art devices comprise electrodes that are in direct contact with surrounding tissue and plaque deposits.

U.S. Pat. No. 6,398,792 to O'Connor discloses an angioplasty catheter with a transducer using a balloon for focusing of ultrasonic energy. The O'Connor device includes two balloons located on the catheter and a transducer located on the catheter, with both the balloons and the transducers located within the vasculature. Since the O'Connor device requires that the transducers be located within the vasculature of the body, procedures are limited to large diameter vasculatures that the transducers can fit into while leaving healthy tissue proximal to the site of an occlusion of plaque susceptible to harm from the large vibrations of the transducer. Furthermore, there is a high risk of damaging the vasculature in the method in which the balloon is inflated.

U.S. Pat. No. 6,464,660 to Brisken et al. discloses balloon catheters having ultrasonically driven interface surfaces and methods for their use. The Brisken et al. device has a catheter comprising a catheter body, a transducer located on the catheter, an inflatable balloon on the catheter and a stent carried over the balloon. The Brisken et al. device requires the transducer be placed within the vasculature of the body and does not break the occlusion down to microscopic sizes to allow for efficient and complete removal of the occlusion during the long treatment procedure. The Brisken et al. device utilizes large forces applied from the inflatable balloon to the inner walls of the vasculature within the body.

U.S. Pat. No. 6,383,151 to Diederich et al. discloses a circumferential ablation device assembly comprising an elongate body, an expandable member around a length of the elongate body and a transducer at a distal end of the elongate body within the expandable member. In the Diederich et al. device, the expandable member is inflated and the transducer located within the expandable member emits energy to ablate the tissue. The Diederich et al. device utilizes an expandable member that compresses the tissue and imparts large forces on the vasculatures in the body. The Diederich et al. device does not deliver energy to the tissue to effectively remove the occlusion.

The prior art devices and methods of removing an occlusion are not effective at delivering energy to the occlusion to remove the occlusive material and have a high risk of damage to the vasculature and other associated health risks to the patient. The prior art devices are complex and require large components be inserted into a vasculature of the body. Therefore, there is a need in the art for an apparatus and a method of removing an occlusion in a vasculature of the body that reduces the treatment time to remove the occlusion, matches the anatomy of a vasculature, does not harm the patient or the vasculature the ultrasonic probe is moving through and increases a radial span of an occlusion destroying effect of an ultrasonic probe.

### SUMMARY OF THE INVENTION

The present invention is an apparatus and method for an ultrasonic medical device with a non-compliant balloon. The present invention is an ultrasonic medical device having an ultrasonic probe having a proximal end, a distal end and a longitudinal axis therebetween. A catheter comprising a proximal end, a distal end and a longitudinal axis therebetween surrounds the ultrasonic probe. The ultrasonic medical device includes an inflation lumen located along the longitudinal axis of the catheter and a balloon is supported by the catheter. An inner surface of the balloon is in communication with the inflation lumen. The balloon is inflated with a medium that engages the inner surface of the balloon.

The present invention is an ultrasonic medical device for ablating an occlusion. The ultrasonic medical device comprises a catheter having a proximal end, a distal end and a longitudinal axis therebetween. A balloon having an outer surface, an inner surface, a proximal end and a distal end engages the catheter along the longitudinal axis of the catheter. An inflation lumen located along the longitudinal axis of the catheter is in communication with the balloon. An elongated ultrasonic probe having a proximal end, a distal end and a longitudinal axis therebetween extends through at least a portion of the longitudinal axis of the catheter.

The present invention provides a method of ablating a biological material in a vasculature of a body. An ultrasonic probe is inserted into a vasculature adjacent to the biological material. A catheter comprising a balloon on an outer surface of the catheter is advanced until the balloon is adjacent to the biological material. The balloon is inflated so at least a portion of an outer surface of the balloon engages the biological material. The ultrasonic probe is energized to produce a transverse ultrasonic vibration that vibrates at least a portion of the balloon to break the biological material into a particulate. The balloon is inflated to a larger diameter to engage the biological material and the ultrasonic probe is energized to ablate the biological material.

The present invention provides a method of removing an occlusion in a vasculature of a body. A biocompatible material member is inserted inside the vasculature and advanced until a portion of the biocompatible material member is adjacent to the occlusion. A flexible ultrasonic probe is moved through the biocompatible material member until the flexible ultrasonic probe is adjacent to the occlusion. An ultrasonic energy source is activated to produce a transverse ultrasonic vibration along at least a portion of a longitudinal axis of the flexible ultrasonic probe that vibrates at least a portion of the biocompatible material member to remove the occlusion.

The present invention is an ultrasonic medical device comprising a non-compliant balloon and an ultrasonic probe used to ablate an occlusion. The balloon expands a treatment area of an occlusion destroying effect of the ultrasonic probe. The present invention provides an ultrasonic medical device with a non-compliant balloon and an ultrasonic probe that is safe, simple, user-friendly, reliable and cost effective.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a side plan view of an ultrasonic medical device of the present invention with an ultrasonic probe surrounded by a catheter that supports a balloon between a proximal end and a distal end of the catheter.

FIG. 2A is a side plan view of an ultrasonic probe of the present invention capable of operating in a transverse mode.

FIG. 2B is a side plan view of an ultrasonic probe of the present invention having an approximately uniform diameter from a proximal end of the ultrasonic probe to a distal end of the ultrasonic probe.

FIG. 3 is a side plan view of a catheter with a balloon supported by the catheter between a proximal end and a distal end of the catheter.

FIG. 4 is a fragmentary side plan view of an ultrasonic medical device of the present invention with a segment of an ultrasonic probe within a catheter that supports a balloon between a proximal end and a distal end of the catheter.

FIG. 5 is a fragmentary side plan view of an ultrasonic medical device of the present invention wherein a balloon that is supported by a catheter is in a deflated state and located adjacent to an occlusion inside a vasculature of a body and a segment of an ultrasonic probe is located within the catheter.

FIG. 6 is a fragmentary side plan view of an ultrasonic medical device of the present invention wherein a balloon that is supported by a catheter is in an inflated state and a portion of an outer surface of the balloon engages an occlusion and a segment of an ultrasonic probe is located within the catheter.

FIG. 7 is a fragmentary side plan view of an ultrasonic medical device of the present invention showing a plurality of transverse nodes and a plurality of transverse anti-nodes along a portion of a longitudinal axis of an ultrasonic probe, the ultrasonic probe surrounded by a catheter and a balloon that is supported by the catheter. The balloon is in an inflated state such that a portion of an outer surface of the balloon engages an occlusion in a vasculature.

FIG. 8 is a computer generated model of a cylindrical mode of vibration of a portion of a balloon.

FIG. 9 is a computer generated model of a transverse mode of vibration of a portion of a balloon.

FIG. 10 is a fragmentary side plan view of an ultrasonic medical device of the present invention wherein a balloon that is supported by a catheter is deflated, a segment of an ultrasonic probe is located within the catheter and an occlusion in a vasculature has been removed.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides an apparatus and a method for using an ultrasonic medical device with a non-compliant balloon. An ultrasonic medical device comprises an ultrasonic probe, a catheter surrounding the ultrasonic probe and a balloon supported by the catheter. In a preferred embodiment of the present invention, the ultrasonic probe, the catheter and the balloon are advanced to a site of an occlusion. The balloon is inflated to a diameter such that an outer surface of the balloon engages the occlusion and an ultrasonic energy source is activated. The ultrasonic energy from the ultrasonic energy source vibrates the ultrasonic probe in a transverse direction, causing at least a portion of the balloon to vibrate to break the occlusion into a particulate. In a preferred embodiment of the present invention, the balloon is repeatedly inflated to a larger diameter and the ultrasonic probe is repeatedly energized to effectively remove the occlusion. The balloon expands a treatment area of an occlusion destroying effect of the ultrasonic probe and increases a radial span of an occlusion destroying effect of the ultrasonic probe.

The following terms and definitions are used herein:

“Ablate” as used herein refers to removing, clearing, destroying or taking away a biological material. “Ablation” as used herein refers to a removal, clearance, destruction, or taking away of the biological material.

“Node” as used herein refers to a region of a minimum energy emitted by an ultrasonic probe at or proximal to a specific location along a longitudinal axis of the ultrasonic probe.

“Anti-node” as used herein refers to a region of a maximum energy emitted by an ultrasonic probe at or proximal to a specific location along a longitudinal axis of the ultrasonic probe.

“Probe” as used herein refers to a device capable of propagating an energy emitted by the ultrasonic energy source along a longitudinal axis of the probe, resolving the energy into an effective cavitational energy at a specific resonance (defined by a plurality of nodes and a plurality of anti-nodes along an “active area” of the probe).

“Transverse” as used herein refers to a vibration of a probe not parallel to a longitudinal axis of the probe. A “transverse wave” as used herein is a wave propagated along the probe in which a direction of a disturbance at a plurality of points of a medium is not parallel to a wave vector.

“Biological material” as used herein refers to a collection of a matter including, but not limited to, a group of similar cells, intravascular blood clots, thrombus, fibrin, occlusions, calcified plaque, calcium deposits, occlusional deposits, atherosclerotic plaque, fatty deposits, adipose tissues, atherosclerotic cholesterol buildup, plaque, fibrous material buildup, arterial stenoses, minerals, high water content tissues, platelets, cellular debris, wastes and other occlusive materials.

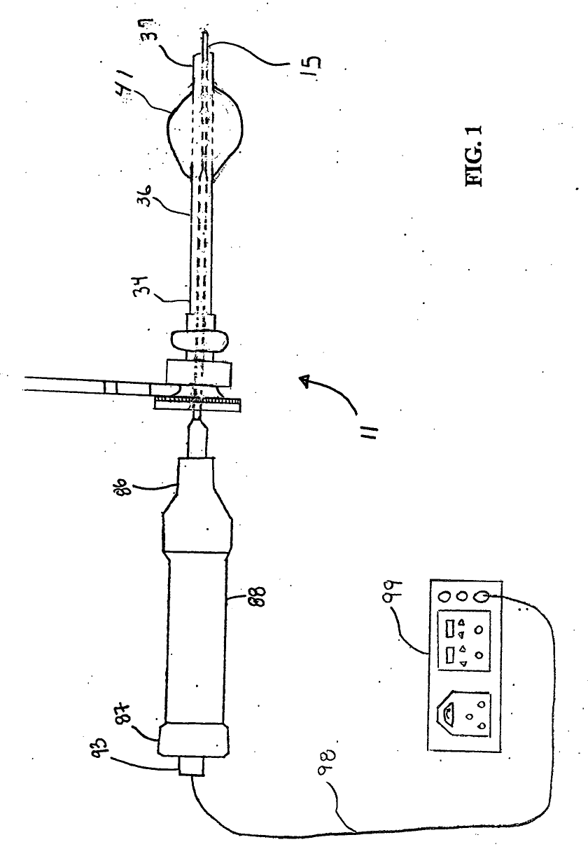

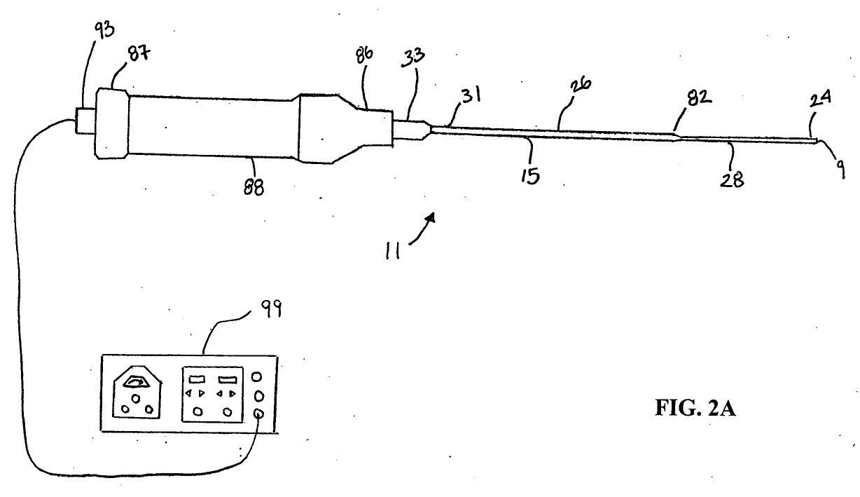

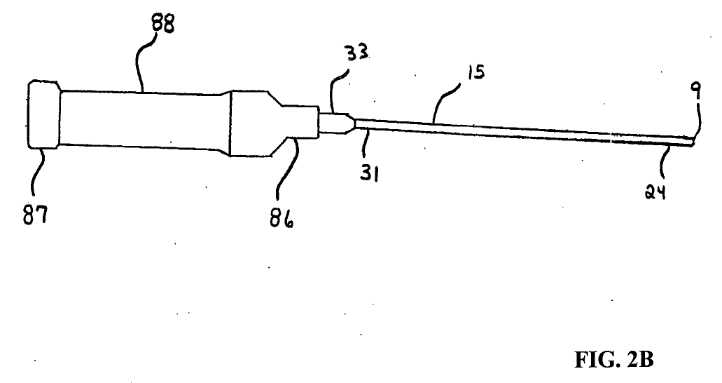

An apparatus for an ultrasonic medical device with a non-compliant balloon of the present invention is illustrated generally at 11 in FIG. 1. FIG. 1 shows an ultrasonic medical device 11 with an ultrasonic probe 15 surrounded by a catheter 36 that supports a balloon 41 between a proximal end 34 and a distal end 37 of the catheter 36. The balloon 41 is in an inflated state and surrounding a portion of a longitudinal axis of the ultrasonic probe 15. The ultrasonic medical device 11 includes the ultrasonic probe 15 and an ultrasonic energy source or generator 99 for the production of an ultrasonic energy. A more detailed representation of an ultrasonic probe 15 is illustrated in FIG. 2A and FIG. 2B. A handle 88, comprising a proximal end 87 and a distal end 86, surrounds a transducer within the handle 88. The transducer having a first end engaging the ultrasonic energy source 99 and a second end engaging a proximal end 31 of the ultrasonic probe 15 transmits an ultrasonic energy to the ultrasonic probe 15. A connector 93 and a connecting wire 98 engage the ultrasonic energy source 99 to the transducer within the handle 88. The ultrasonic probe 15 includes the proximal end 31, a distal end 24 and a longitudinal axis between the proximal end 31 and the distal end 24. As shown in FIG. 2A, a diameter of the ultrasonic probe 15 decreases from a first defined interval 26 to a second defined interval 28 along the longitudinal axis of the ultrasonic probe 15 over an at least one transition 82. At the distal end 24 of the longitudinal axis of the ultrasonic probe 15, the ultrasonic probe 15 ends in a probe tip 9. A coupling 33 that engages the proximal end 31 of the ultrasonic probe 15 to the transducer within the handle 88 is illustrated generally in FIGS. 1, 2A and 2B. In a preferred embodiment of the present invention, the coupling 33 is a quick attachment-detachment system. An ultrasonic medical device with a rapid attachment and detachment means is described in the Assignee's U.S. Pat. No. 6,695,782 and Assignee's co-pending patent applications U.S. Ser. No. 10/268,487 and U.S. Ser. No. 10/268,843, which further describe the quick attachment-detachment system and the entirety of these applications are hereby incorporated herein by reference.

FIG. 2B shows an alternative embodiment of the ultrasonic probe 15 of the present invention. In the embodiment of the present invention shown in FIG. 2B, the diameter of the ultrasonic probe 15 is approximately uniform from the proximal end 31 of the ultrasonic probe 15 to the distal end 24 of the ultrasonic probe 15.

The ultrasonic probe 15 has a stiffness that gives the ultrasonic probe 15 a flexibility so it can be articulated in a vasculature of a body. The ultrasonic probe 15 has a stiffness that gives the ultrasonic probe 15 a flexibility allowing the ultrasonic probe 15 to be deflected, flexed and bent through the tortuous paths of the vasculature. The ultrasonic probe 15 can be bent, flexed and deflected to reach the biological material that would otherwise be difficult to reach.

In a preferred embodiment of the present invention, the ultrasonic probe 15 is a wire. In another embodiment of the present invention, the ultrasonic probe 15 is elongated. In a preferred embodiment of the present invention, the diameter of the ultrasonic probe 15 decreases from the first defined interval 26 to the second defined interval 28. In another embodiment of the present invention, the diameter of the ultrasonic probe 15 decreases at greater than two defined intervals. In a preferred embodiment of the present invention, the transitions 82 of the ultrasonic probe 15 are tapered to gradually change the diameter from the proximal end 31 to the distal end 24 along the longitudinal axis of the ultrasonic probe 15. In another embodiment of the present invention, the transitions 82 of the ultrasonic probe 15 are stepwise to change the diameter from the proximal end 31 to the distal end 24 along the longitudinal axis of the ultrasonic probe 15. Those skilled in the art will recognize that there can be any number of defined intervals and transitions, and that the transitions can be of any shape known in the art and be within the spirit and scope of the present invention.

The probe tip 9 can be any shape including, but not limited to, bent, rounded, a ball or larger shapes. In a preferred embodiment of the present invention, the probe tip 9 is smooth to prevent damage to the vasculature. In one embodiment of the present invention, the ultrasonic energy source 99 is a physical part of the ultrasonic medical device 11. In another embodiment of the present invention, the ultrasonic energy source 99 is not a physical part of the ultrasonic medical device 11.

In a preferred embodiment of the present invention, the cross section of the ultrasonic probe 15 is approximately circular. In other embodiments of the present invention, a shape of the cross section of the ultrasonic probe 15 includes, but is not limited to, square, trapezoidal, oval, triangular, circular with a flat spot and similar cross sections. Those skilled in the art will recognize that other cross sectional geometric configurations known in the art would be within the spirit and scope of the present invention.

The ultrasonic probe 15 is inserted into the vasculature and may be disposed of after use. In a preferred embodiment of the present invention, the ultrasonic probe 15 is for a single use and on a single patient. In a preferred embodiment of the present invention, the ultrasonic probe 15 is disposable. In another embodiment of the present invention, the ultrasonic probe 15 can be used multiple times.

In a preferred embodiment of the present invention, the ultrasonic probe 15 comprises titanium or a titanium alloy. In a preferred embodiment of the present invention, the ultrasonic probe comprises Ti-6Al-4V. The elements comprising Ti-6Al-4V and the representative elemental weight percentages of Ti-6Al-4V are titanium (about 90%), aluminum (about 6%), vanadium (about 4%), iron (maximum about 0.25%) and oxygen (maximum about 0.2%). Titanium is strong, flexible, low density, and easily fabricated metal that is used as a structural material. Titanium and its alloys have excellent corrosion resistance in many environments and have good elevated temperature properties. In another embodiment of the present invention, the ultrasonic probe 15 comprises stainless steel. In another embodiment of the present invention, the ultrasonic probe 15 comprises an alloy of stainless steel. In another embodiment of the present invention, the ultrasonic probe 15 comprises aluminum. In another embodiment of the present invention, the ultrasonic probe 15 comprises an alloy of aluminum. In another embodiment of the present invention, the ultrasonic probe 15 comprises a combination of titanium and stainless steel. Those skilled in the art will recognize that the ultrasonic probe can be comprised of many materials known in the art and be within the spirit and scope of the present invention.

In a preferred embodiment of the present invention, the ultrasonic probe 15 has a small diameter. In a preferred embodiment of the present invention, the diameter of the ultrasonic probe 15 gradually decreases from the proximal end 31 to the distal end 24. In an embodiment of the present invention, the diameter of the distal end 24 of the ultrasonic probe 15 is about 0.004 inches. In another embodiment of the present invention, the diameter of the distal end 24 of the ultrasonic probe 15 is about 0.015 inches. In other embodiments of the present invention, the diameter of the distal end 24 of the ultrasonic probe 15 varies between about 0.003 inches and about 0.025 inches. Those skilled in the art will recognize an ultrasonic probe 15 can have a diameter at the distal end 24 smaller than about 0.003 inches, larger than about 0.025 inches, and between about 0.003 inches and about 0.025 inches and be within the spirit and scope of the present invention.

In an embodiment of the present invention, the diameter of the proximal end 31 of the ultrasonic probe 15 is about 0.012 inches. In another embodiment of the present invention, the diameter of the proximal end 31 of the ultrasonic probe 15 is about 0.025 inches. In other embodiments of the present invention, the diameter of the proximal end 31 of the ultrasonic probe 15 varies between about 0.003 inches and about 0.025 inches. Those skilled in the art will recognize the ultrasonic probe 15 can have a diameter at the proximal end 31 smaller than about 0.003 inches, larger than about 0.025 inches, and between about 0.003 inches and about 0.025 inches and be within the spirit and scope of the present invention.

In an embodiment of the present invention, the diameter of the ultrasonic probe 15 is approximately uniform from the proximal end 31 to the distal end 24 of the ultrasonic probe 15. In another embodiment of the present invention, the diameter of the ultrasonic probe 15 gradually decreases from the proximal end 31 to the distal end 24. In an embodiment of the present invention, the ultrasonic probe 15 may resemble a wire. In an embodiment of the present invention, the gradual change of the diameter from the proximal end 31 to the distal end 24 occurs over the at least one transition 82 with each transition 82 having an approximately equal length. In another embodiment of the present invention, the gradual change of the diameter from the proximal end 31 to the distal end 24 occurs over a plurality of transitions 82 with each transition 82 having a varying length. The transition 82 refers to a section where the diameter varies from a first diameter to a second diameter.

The physical properties (i.e., length, cross sectional shape, dimensions, etc.) and material properties (i.e., yield strength, modulus, etc.) of the ultrasonic probe 15 are selected for operation of the ultrasonic probe 15 in the transverse mode. The length of the ultrasonic probe 15 of the present invention is chosen so as to be resonant in a transverse mode. In an embodiment of the present invention, the ultrasonic probe 15 is between about 30 centimeters and about 300 centimeters in length. In an embodiment of the present invention, the ultrasonic probe 15 is a wire. Those skilled in the art will recognize an ultrasonic probe can have a length shorter than about 30 centimeters and a length longer than about 300 centimeters and be within the spirit and scope of the present invention.

The handle 88 surrounds the transducer located between the proximal end 31 of the ultrasonic probe 15 and the connector 93. In a preferred embodiment of the present invention, the transducer includes, but is not limited to, a horn, an electrode, an insulator, a backnut, a washer, a piezo microphone, and a piezo drive. The transducer is capable of an acoustic impedance transformation of electrical energy provided by the ultrasonic energy source 99 to mechanical energy. The transducer sets the operating frequency of the ultrasonic medical device 11. The transducer transmits ultrasonic energy received from the ultrasonic energy source 99 to the ultrasonic probe 15. Energy from the ultrasonic energy source 99 is transmitted along the longitudinal axis of the ultrasonic probe 15, causing the ultrasonic probe 15 to vibrate in a transverse mode. The transducer is capable of engaging the ultrasonic probe 15 at the proximal end 31 with sufficient restraint to form an acoustical mass that can propagate the ultrasonic energy provided by the ultrasonic energy source 99.

The ultrasonic energy source 99 produces a transverse ultrasonic vibration along a portion of the longitudinal axis of the ultrasonic probe 15. The ultrasonic probe 15 can support the transverse ultrasonic vibration along the longitudinal axis of the ultrasonic probe 15. The transverse mode of vibration of the ultrasonic probe 15 according to the present invention differs from an axial (or longitudinal) mode of vibration disclosed in the prior art. Rather than vibrating in an axial direction, the ultrasonic probe 15 of the present invention vibrates in a direction transverse (not parallel) to the axial direction. As a consequence of the transverse vibration of the ultrasonic probe 15, the occlusion destroying effects of the ultrasonic medical device 11 are not limited to those regions of the ultrasonic probe 15 that may come into contact with the occlusion 16. Rather, as a section of the longitudinal axis of the ultrasonic probe 15 is positioned in proximity to an occlusion, a diseased area or lesion, the occlusion 16 is removed in all areas adjacent to a plurality of energetic transverse nodes and transverse anti-nodes that are produced along a portion of the longitudinal axis of the ultrasonic probe 15. Transversely vibrating ultrasonic probes for biological material ablation are described in the Assignee's U.S. Pat. No. 6,551,337; U.S. Pat. No. 6,652,547; U.S. Pat. No. 6,695,781; and U.S. Pat. No. 6,660,013 which further describe the design parameters for such an ultrasonic probe and its use in ultrasonic devices for an ablation, and the entirety of these patents and patent applications are hereby incorporated herein by reference.

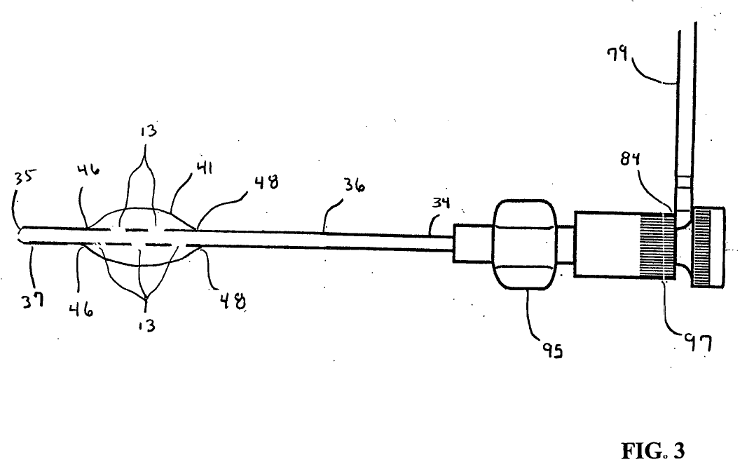

FIG. 3 shows a catheter 36 with a balloon 41 supported by the catheter 36 between a proximal end 34 and a distal end 37 of the catheter 36. The catheter 36 comprises a catheter tip 35 at the distal end 37 of the catheter 36 and a plurality of fenestrations 13 along a longitudinal axis of the catheter 36. The balloon 41 engages the catheter 36 at an at least one engagement position along the longitudinal axis of the catheter 36. In a preferred embodiment of the present invention, the balloon 41 engages the catheter 36 at a first engagement position 48 and a second engagement position 46 located on the longitudinal axis of the catheter 36. The balloon 41 engages the catheter 36 in a manner known in the art. In the embodiment of the present invention shown in FIG. 3, the catheter 36 includes a port 84, one or more placement wings 95 and one or more valves 97. A connective tubing 79 engages the catheter 36 at the port 84 and the connective tubing 79 can be opened or closed with one or more valves 97. The connective tubing 79 is used to deliver a medium to inflate the balloon 41. The catheter 36 comprises the one or more placement wings 95 to assist in the placement of the catheter 36.

The catheter 36 is a thin, flexible, hollow tube that is small enough to be threaded through a vein or an artery. The catheter 36 can be used for several purposes in a surgical procedure. The catheter 36 can be used to deliver various medical devices both outside of an interior of the catheter 36 and inside of the catheter 36. The catheter 36 can be used to deliver fluids into or withdraw fluids from a body. Patients generally do not feel the movement of the catheter 36 through their body. Once in place, the catheter 36 allows a number of tests or other treatment procedures to be performed. Those skilled in the art will recognize that many catheters known in the art can be used with the present invention and still be within the spirit and scope of the present invention.

In one embodiment of the present invention, the catheter comprises polytetrafluoroethylene (PTFE). In another embodiment of the present invention, the catheter comprises latex. In other embodiments of the present invention, the catheter comprises a material including, but not limited to, rubber, silicone, teflon, platinum and similar materials. Those skilled in the art will recognize that catheter may comprise many other materials and still be within the spirit and scope of the present invention.

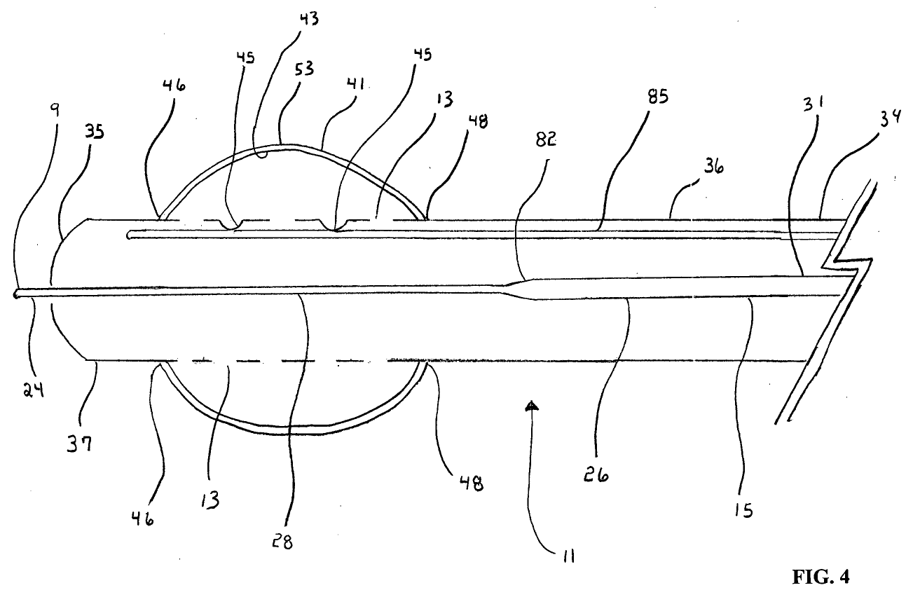

FIG. 4 shows a fragmentary view of the ultrasonic medical device 11 with the catheter 36, the balloon 41 supported by the catheter 36 and the ultrasonic probe 15 within the interior of the catheter 36. In an embodiment of the present invention shown in FIG. 4, the tip 9 of the ultrasonic probe 15 extends past the distal end 37 of the catheter 36. In another embodiment of the present invention, the tip 9 of the ultrasonic probe 15 is located within the interior of the catheter 36 and does not extend past the catheter tip 35. The ultrasonic medical device 11 includes an inflation lumen 85 and an at least one inflation opening 45 in the inflation lumen 85. The balloon 41 comprises an outer surface 53 and an inner surface 43.

The inflation lumen 85 is used to deliver a medium from an inflation mechanism to inflate the balloon 41. In a preferred embodiment of the present invention, the medium is a liquid medium. The medium moves along the insertion lumen 85 and through at least one inflation opening 45 where the medium engages the inner surface 43 of the balloon 41, where the inner surface 43 of the balloon 41 is in communication with the inflation lumen 85. In a preferred embodiment of the present invention shown in FIG. 4, the inflation lumen 85 is located within the interior of the catheter 36. In another embodiment of the present invention, the inflation lumen 85 is located outside of the catheter 36. In a preferred embodiment of the present invention, the balloon 41 is inflated by providing a liquid medium through the inflation lumen 85. In a preferred embodiment of the present invention, the medium is a contrast mixed with water. In another embodiment of the present invention, the medium is saline. In another embodiment of the present invention, the medium is a gas. Those skilled in the art will recognize there are many mediums used to inflate a balloon known in the art that can be used with the present invention and still be within the spirit and scope of the present invention.

An inflation mechanism is used to provide the medium into the connective tubing 79 to inflate the balloon 41 to a desired size and pressure. The medium flows along a longitudinal axis within the inflation lumen 85 and the medium moves through the at least one inflation opening 45. The balloon 41 is inflated as the medium engages the inner surface 43 of the balloon 41 and expands the balloon 41. Inflation mechanisms include, but are not limited to, syringes, screw mounted hydraulic syringes and similar devices. Those skilled in the art will recognize there are several inflation mechanisms and methods of inserting a medium into an inflation lumen known in the art that are within the spirit and scope of the present invention.

In a preferred embodiment of the present invention, the balloon 41 is a non-compliant balloon. Balloon compliance is defined as the ability of the balloon 41 to expand in diameter at various inflation pressures. In traditional balloon angioplasty procedures where a balloon is used to compress an occlusion into a wall of the vasculature, the compliance of the balloon affects the performance of the balloon 41 when compressing an occlusion. A non-compliant balloon maintains its size and shape, even when inflated at high pressures. Non-compliant balloons will change diameter by approximately 3% when inflated. Non-compliant materials include, but are not limited to, polyethylene terephthalate (PET), polyurethane with nylon, duralyin and similar materials. Those skilled in the art will recognize there are many non-compliant materials known in the art that would be within the spirit and scope of the present invention.

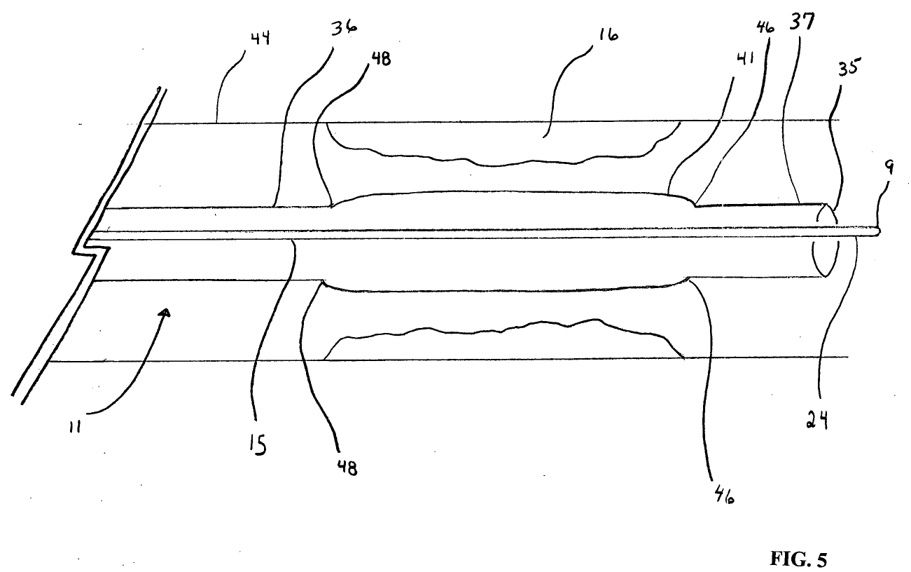

FIG. 5 shows a fragmentary view of the ultrasonic medical device 11 located adjacent to an occlusion 16 inside a vasculature 44 of a body. FIG. 5 illustrates the balloon 41 in a deflated state, where the balloon 41 is supported by the catheter 36 and at least a portion of an outer surface of the balloon 41 is adjacent to the occlusion 16. In a preferred embodiment of the present invention, the occlusion 16 comprises a biological material. The ultrasonic probe 15 extends through the interior of the catheter 36 and a section of the longitudinal axis of the ultrasonic probe 15, including the probe tip 9, extends past the catheter tip 35. FIG. 5 shows a step in a procedure of removing the occlusion 16 where the components of the ultrasonic medical device are at the site of the occlusion 16. Several steps precede the state shown in FIG. 5, and these steps will be discussed below.

In a preferred embodiment of the present invention, the ultrasonic probe 15 is inserted into the vasculature 44 and moved adjacent to the site of the occlusion 16. A guide catheter is placed over the proximal end 31 of the ultrasonic probe 15 and moved along the longitudinal axis of the ultrasonic probe 15. The catheter 36, with the balloon 41 that is supported by the catheter 36 and the inflation lumen 85 (not shown in FIG. 5) within the catheter 36, is moved over the proximal end 31 of the ultrasonic probe 15 and moved along the longitudinal axis of the ultrasonic probe 15 until the balloon 41 is adjacent to the occlusion 16. Those skilled in the art will recognize there are several ways to deliver a catheter, a balloon supported by the catheter and an ultrasonic probe within the catheter that are known in the art that are within the spirit and scope of the present invention.

In another embodiment of the present invention, a biocompatible material member is moved within the guide catheter and advanced in the vasculature until a portion of the biocompatible material member is adjacent to the occlusion. The ultrasonic probe 15 is moved through the biocompatible material member until the ultrasonic probe 15 is adjacent to the occlusion 16. The biocompatible material member includes, but is not limited to, a catheter, a balloon, a sheath, a heat shrink and similar members. Those skilled in the art will recognize that other biocompatible material members known in the art would be within the spirit and scope of the present invention.

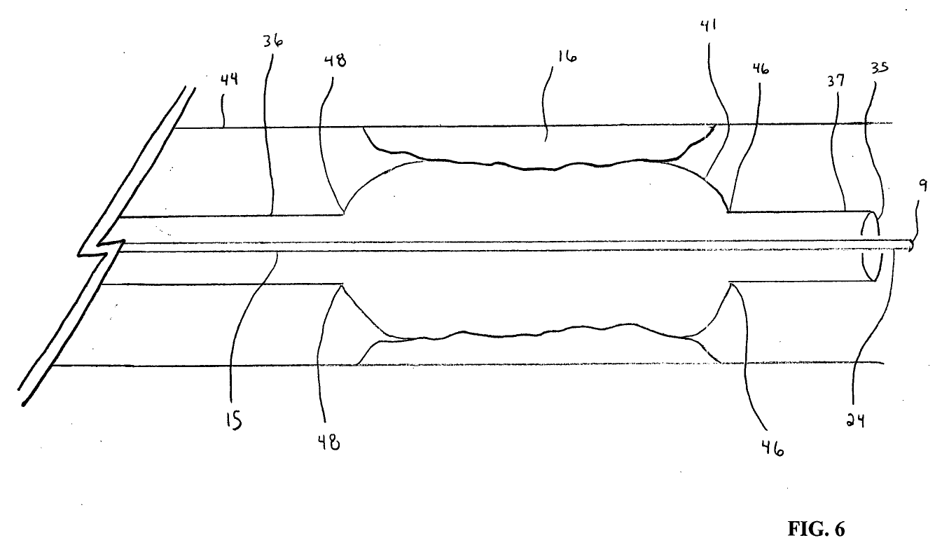

FIG. 6 shows a fragmentary side plan view of the ultrasonic medical device 11 wherein the balloon 41 is inflated and at least a portion of an outer surface 53 of the balloon 41 engages the occlusion 16. The medium is injected from the inflation mechanism into the connective tubing 79, moves along the inside of the inflation lumen 85 and through the at least one inflation opening 45. The medium engages the inner surface 43 of the balloon 41 and the balloon 41 is inflated to a size wherein at least a portion of the outer surface 53 of the balloon 41 engages the occlusion 16. The balloon 41, upon inflation, is a generally oval-shaped balloon having the first engagement position 48 and the second engagement position 46. Since the balloon 41 is oval-shaped, the balloon 41 has a large surface area which engages the occlusion 16 upon inflation. Unlike traditional angioplasty procedures where the angioplasty balloon is blown up to high pressures, the balloon 41 of the present invention is inflated to a low pressure that does not stretch the walls of the vasculature 44 and does not compress the occlusion 16. In traditional angioplasty procedures, the vasculature is subjected to high mechanical forces and the occlusion is compressed into the walls of the vasculature.

As shown in FIG. 6, with the balloon 41 inflated to a pressure in which the outer surface 53 of the balloon 41 engages the occlusion 16, the ultrasonic energy source 99 is activated to energize the ultrasonic probe 15. The ultrasonic energy source 99 provides a low power electric signal of between about 2 watts to about 15 watts to the transducer that is located within the handle 88. The transducer converts electrical energy provided by the ultrasonic energy source 99 to mechanical energy. The operating frequency of the ultrasonic medical device 11 is set by the transducer and the ultrasonic energy source 99 finds the resonant frequency of the transducer through a Phase Lock Loop. By an appropriately oriented and driven cylindrical array of piezoelectric crystals of the transducer, the horn creates a longitudinal wave along at least a portion of the longitudinal axis of the ultrasonic probe 15. The longitudinal wave is converted to a transverse wave along at least a portion of the longitudinal axis of the ultrasonic probe 15 through a nonlinear dynamic buckling of the ultrasonic probe 15.

As the transverse wave is transmitted along the longitudinal axis of the ultrasonic probe 15, a transverse ultrasonic vibration is created along the longitudinal axis of the ultrasonic probe 15. The ultrasonic probe 15 is vibrated in a transverse mode of vibration. The transverse mode of vibration of the ultrasonic probe 15 differs from an axial (or longitudinal) mode of vibration disclosed in the prior art. The transverse ultrasonic vibrations along the longitudinal axis of the ultrasonic probe 15 create a plurality of transverse nodes and a plurality of transverse anti-nodes along a portion of the longitudinal axis of the ultrasonic probe 15.

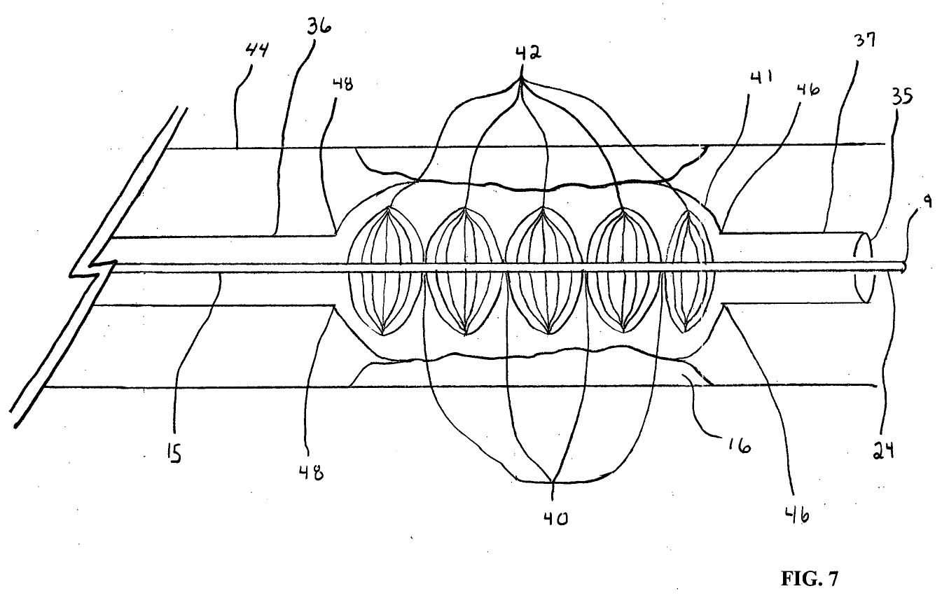

FIG. 7 shows a fragmentary side plan view of the ultrasonic medical device 11 of the present invention showing a plurality of transverse nodes 40 and a plurality of transverse anti-nodes 42 along a portion of the longitudinal axis of the ultrasonic probe 15. The transverse nodes 40 are areas of minimum energy and minimum vibration. The transverse anti-nodes 42, or areas of maximum energy and maximum vibration, also occur at repeating intervals along the portion of the longitudinal axis of the ultrasonic probe 15. The number of transverse nodes 40 and transverse anti-nodes 42, and the spacing of the transverse nodes 40 and transverse anti-nodes 42 of the ultrasonic probe 15 depend on the frequency of energy produced by the ultrasonic energy source 99. The separation of the transverse nodes 40 and transverse anti-nodes 42 is a function of the frequency, and can be affected by tuning the ultrasonic probe 15. In a properly tuned ultrasonic probe 15, the transverse anti-nodes 42 will be found at a position approximately one half of the distance between the transverse nodes 40 located adjacent to each side of the transverse anti-nodes 42.

In a preferred embodiment of the present invention, the transverse ultrasonic vibration of the ultrasonic probe 15 vibrates at least a portion of the balloon 41. The transverse wave is transmitted along the longitudinal axis of the ultrasonic probe 15 and the interaction of the surface of the ultrasonic probe 15 with the medium surrounding the ultrasonic probe creates a pressure wave that is transmitted to the balloon 41. As the transverse wave is transmitted along the longitudinal axis of the ultrasonic probe 15, the ultrasonic probe vibrates transversely. The transverse motion of the ultrasonic probe 15 produces cavitation in the medium surrounding the ultrasonic probe 15 and the balloon 41. Cavitation is a process in which small voids are formed in a surrounding medium through the rapid motion of the ultrasonic probe 15 and the voids are subsequently forced to compress. The compression of the voids creates a wave of acoustic energy which acts to dissolve the matrix binding the occlusion, while having no damaging effects on healthy tissue.

With at least a portion of the outer surface 53 of the balloon 41 engaging the occlusion 16, the acoustic pressure contour is created circumferentially around the balloon 41, where the acoustic energy from the acoustic pressure contour is transmitted to the occlusion 16. As a portion of the balloon 41 vibrates, the balloon 41 focuses the occlusion destroying effects of the ultrasonic probe 15 to resolve the occlusion 16 to a particulate comparable in size to red blood cells (about 5 microns in diameter).

The occlusion 16 is resolved into a particulate having a size on the order of red blood cells (approximately 5 microns in diameter). The size of the particulate is such that the particulate is easily discharged from the body through conventional methods or simply dissolves into the blood stream. A conventional method of discharging the particulate from the body includes transferring the particulate through the blood stream to the kidney where the particulate is excreted as bodily waste.

In a preferred embodiment of the present invention, the balloon 41 vibrates in a transverse mode of vibration and a cylindrical mode of vibration. Energy is transferred to the occlusion 16 in the transverse mode of vibration and the cylindrical mode of vibration. In another embodiment of the present invention, the balloon 41 vibrates in a cylindrical mode of vibration. In another embodiment of the present invention, the balloon 41 operates in a transverse mode of vibration. In the cylindrical mode of vibration, the surface of the balloon 41 acts like a membrane that alternately vibrates up and down. In the transverse mode of vibration, the balloon vibrates up and down in the transverse direction.

In a preferred embodiment of the present invention, the resonant frequency of the balloon 41 is similar to the resonant frequency of the ultrasonic probe 15, creating a coupled resonance. When the resonant frequency of the balloon 41 is approximately equal to the resonant frequency of the ultrasonic probe 15, the balloon 41 is excited in unison with the ultrasonic probe 15, maximizing the amplitude of vibration of the balloon/ultrasonic probe system and producing the largest amplitude of vibration of the balloon 41. The amplitude of vibration of a system is maximized when the forcing frequency is at the resonance frequency. The ultrasonic energy transferred from the ultrasonic probe 15 is transferred to the balloon 41 where it is transferred to the occlusion 16.

As stated previously, in the preferred embodiment of the present invention, the balloon 41 vibrates in a transverse mode of vibration and a cylindrical mode of vibration. The balloon 41 vibrating in a transverse mode of vibration and a cylindrical mode of vibration occurs when the frequency of vibration of the ultrasonic probe 15 is close to both a resonant frequency of a cylindrical mode of vibration of the balloon 41 and a resonant frequency of a transverse mode of vibration for the balloon 41. For thin structures, such as the balloon 41, the resonant frequencies of the transverse mode of vibration and the cylindrical mode of vibration are closely spaced, creating an overlap of the cylindrical and transverse modes of vibration.

In another embodiment of the present invention, the balloon 41 vibrates in a cylindrical mode of vibration. When the frequency of the transverse vibration of the ultrasonic probe 15 is close to the resonant frequency of the cylindrical mode of vibration of the balloon 41, the cylindrical mode of vibration is excited. Physically, this can be accomplished through one of two mechanisms. First, this can be accomplished by contact of the vibrating ultrasonic probe 15 with the surface of the balloon 41. Second, this can be accomplished by the transmission of the vibrations of the ultrasonic probe 15 to the surface of the balloon 41 via a medium inside of the balloon 41.

In another embodiment of the present invention, the balloon 41 operates in a transverse mode of vibration. When the frequency of the transverse vibration of the ultrasonic probe 15 is close to the resonant frequency of a transverse wave for the balloon 41, the balloon 41 operates in the transverse mode of vibration.

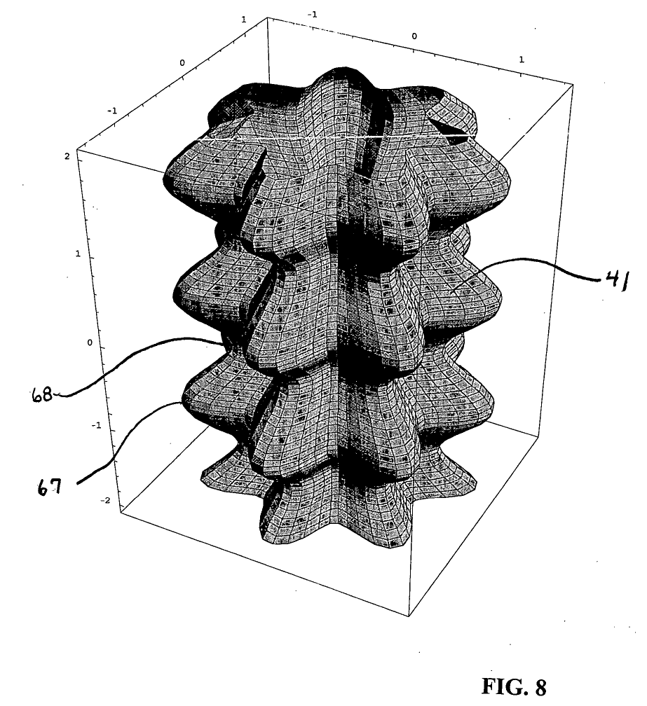

FIG. 8 is a computer generated model of a cylindrical mode of vibration of a portion of the balloon 41. For the balloon 41 vibrating in the cylindrical mode of vibration, the surface of the balloon 41 acts like a membrane, similar to a water surface of the ocean in which the surface oscillates up and down. Referring to FIG. 8, the surface of the balloon 41 has waves circumferentially around, where the surface of the balloon 41 is moved in a two dimensional wave-like manner. As the frequency of the membrane vibrations increase, the spacing between the crests 67 and the troughs 68 of the wave decreases.

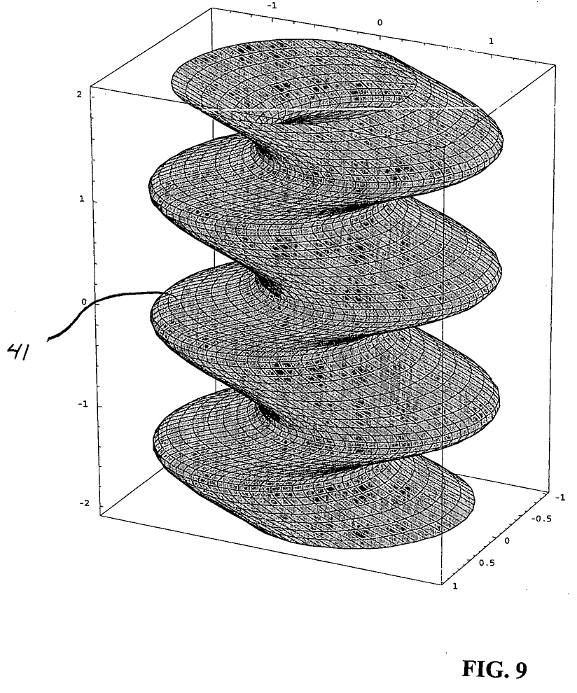

FIG. 9 is a computer generated model of a transverse mode of vibration of a portion of the balloon 41. For the balloon 41 vibrating in the transverse mode of vibration, the whole balloon 41 acts like a rod vibrating in a transverse mode. Referring to FIG. 9, the entire balloon 41 is moving in a one dimensional wave-like manner. As the frequency of the balloon vibrations increase, the wavelength of the transverse waves decrease.

The walls of the balloon 41 are resonated with the energy provided by the transversely vibrating ultrasonic probe 15. The balloon 41 expands a treatment area of an occlusion destroying effect of the ultrasonic probe 15 by increasing a radial span of the acoustic energy that is created circumferentially around the longitudinal axis of the ultrasonic probe 15. With at least a portion of the balloon 41 engaging the occlusion 16, the increased radial span of the occlusion destroying effect of the ultrasonic probe 15 resolves the occlusion 16 into a particulate comparable in size to red blood cells. The occlusion 16 is not compressed into the walls of the vasculature 44 and the vasculature 44 is not subjected to high mechanical forces. The balloon 41 adapts to a contour of the vasculature 44. The particulate is easily discharged from the body through conventional ways or simply dissolves into the blood stream. A conventional way of discharging the particulate from the body includes transferring the particulate through the blood stream to the kidney where the particulate is excreted as bodily waste.

As the occlusion 16 is resolved into particulate that is discharged through the body, the outer surface 53 of the balloon 41 in the initial inflated state is disengaged from the occlusion 16 as the diameter of the vasculature 44 at the occlusion 16 increases. In order for the outer surface 53 of the balloon 41 to engage the residual occlusion 16, the balloon 41 is inflated to a larger diameter. The ultrasonic energy source 99 is activated and the transverse ultrasonic vibration of the ultrasonic probe 15 vibrates at least a portion of the balloon 41 and through a process of cavitation, acoustic energy is generated in the surrounding fluid. As the walls of the balloon 41 vibrate, acoustic energy is transmitted to the occlusion 16 from the vibrating balloon 41. The residual occlusion 16 is resolved into particulate that is easily discharged from the body in conventional ways.

In an embodiment of the present invention, the process of inflating the balloon 41 to a larger diameter and activating the ultrasonic energy source 99 is repeated several times to remove the occlusion 16 from the vasculature 44. Repeatedly inflating the balloon 41 to a larger diameter to engage the residual occlusion 16 and activating the ultrasonic energy source 99 allows for effective removal of the occlusion 16 in a time efficient manner that reduces the risk of occlusion reformation both at the site of the occlusion 16 and downstream of the site of the occlusion 16.

In another embodiment of the present invention, the transverse ultrasonic vibration of the ultrasonic probe 15 moves through the balloon 41 without stimulating vibration of the balloon 41. The balloon 41 serves as a medium through which the transverse ultrasonic vibration is transmitted. Through a process of cavitation, acoustic energy is generated in the surrounding fluid. The pressure wave is created circumferentially around the ultrasonic probe 15 where the acoustic energy from the acoustic pressure contour is transmitted to the occlusion 16. The occlusion destroying effects of the ultrasonic probe 15 resolve the occlusion to a particulate comparable in size to red blood cells (about 5 microns in diameter).

The extent of the acoustic energy is such that the acoustic energy extends radially outward from the longitudinal axis of the ultrasonic probe 15 at the transverse anti-nodes 42 along the portion of the longitudinal axis of the ultrasonic probe 15. In this way, actual treatment time using the transverse mode ultrasonic medical device 11 according to the present invention is greatly reduced as compared to methods disclosed in the prior art that primarily utilize longitudinal vibration (along the axis of the ultrasonic probe). A distinguishing feature of the present invention is the ability to utilize ultrasonic probes 15 of extremely small diameter compared to prior art probes.

The number of transverse nodes 40 and transverse anti-nodes 42 occurring along the longitudinal axis of the ultrasonic probe 15 is modulated by changing the frequency of energy supplied by the ultrasonic energy source 99. The exact frequency, however, is not critical and the ultrasonic energy source 99 run at, for example, about 20 kHz is sufficient to create an effective number of occlusion 16 destroying transverse anti-nodes 42 along the longitudinal axis of the ultrasonic probe 15. The low frequency requirement of the present invention is a further advantage in that the low frequency requirement leads to less damage to healthy tissue. Those skilled in the art understand it is possible to adjust the dimensions of the ultrasonic probe 15, including diameter, length and distance to the ultrasonic energy source 99, in order to affect the number and spacing of the transverse nodes 40 and transverse anti-nodes 42 along a portion of the longitudinal axis of the ultrasonic probe 15.

The present invention allows the use of ultrasonic energy to be applied to the occlusion 16 selectively, because the ultrasonic probe 15 conducts energy across a frequency range from about 10 kHz through about 100 kHz. The amount of ultrasonic energy to be applied to a particular treatment site is a function of the amplitude and frequency of vibration of the ultrasonic probe 15. In general, the amplitude or throw rate of the energy is in the range of about 25 microns to about 250 microns, and the frequency in the range of about 10 kHz to about 100 kHz. In a preferred embodiment of the present invention, the frequency of ultrasonic energy is from about 20 kHz to about 35 kHz. Frequencies in this range are specifically destructive of occlusions 16 including, but not limited to, hydrated (water-laden) tissues such as endothelial tissues, while substantially ineffective toward high-collagen connective tissue, or other fibrous tissues including, but not limited to, vascular tissues, epidermal, or muscle tissues.

In another embodiment of the present invention, a second balloon is located along the longitudinal axis of the catheter 36. With an occlusion 16 that spans a long part of the vasculature 44, the use of a second balloon can expand a treatment area of an occlusion destroying effect of the ultrasonic probe 15. Those skilled in the art will recognize that any number of balloons can be located on the catheter to remove an occlusion and still be within the spirit and scope of the present invention.

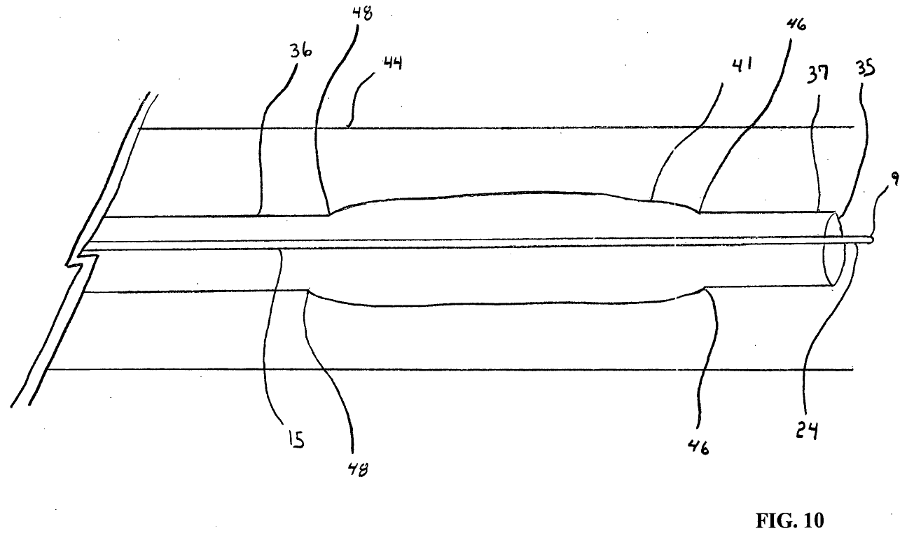

FIG. 10 shows a fragmentary side plan view of the ultrasonic medical device 11 of the present invention wherein the balloon 41 is deflated and the occlusion 16 is removed from the inside walls of the vasculature 44. FIG. 10 illustrates the result of the occlusion ablation process which may require several iterations of successively inflating the balloon to a larger diameter to engage the occlusion 16 and activating the ultrasonic energy source 99. By effectively removing the occlusion 16, the risk of subsequent occlusion reformation is minimized, thereby reducing the health risk to the patient.

The present invention provides a method of ablating an occlusion in the vasculature 44 of the body. The ultrasonic probe 15 is inserted into the vasculature 44 adjacent to the occlusion 16. A catheter 36 comprising a balloon 41 on the outer surface 53 of the catheter 36 is advanced over the proximal end 31 of the ultrasonic probe 15 and along the longitudinal axis of the ultrasonic probe 15 until the balloon 41 is adjacent to the occlusion 16. The balloon is inflated with a medium so at least a portion of the outer surface 53 of the balloon 41 engages the occlusion 16. The ultrasonic energy source 99 is energized to produce a transverse ultrasonic vibration that vibrates at least a portion of the balloon 41, wherein through a process of cavitation generated in the surrounding fluid, acoustic energy breaks the occlusion into a particulate that can be discharged from the body through standard methods. As a portion of the occlusion 16 is removed, the balloon is inflated to a larger diameter with the medium so at least a portion of the outer surface 53 of the balloon 41 engages the occlusion 16 and the ultrasonic energy source 99 is energized.

The balloon 41 concentrates the transverse ultrasonic energy from the ultrasonic probe 15 to the occlusion 16. The balloon 41 expands a treatment area of the occlusion destroying effect of the ultrasonic probe 15 by increasing a radial span of the occlusion destroying effect of the ultrasonic probe 15. The transverse vibration of the outer surface 53 of the balloon 41 resolves the occlusion 16 into particulate in a time efficient manner that prevents health risks to the patient.

The present invention also provides a method of removing the occlusion 16 in the vasculature 44 of the body by inserting the biocompatible material member inside the vasculature 44. The biocompatible material member is advanced adjacent to the occlusion 16 and the ultrasonic probe 15 is moved through the inside of the biocompatible material member until the ultrasonic probe 15 is adjacent to the occlusion 16. The ultrasonic energy source 99 is activated to produce a transverse ultrasonic vibration along at least a portion of the longitudinal axis of the ultrasonic probe 15 that vibrates at least a portion of the biocompatible material member to remove the occlusion 16.

In an alternative embodiment of the present invention, the ultrasonic probe 15 is vibrated in a torsional mode. In the torsional mode of vibration, a portion of the longitudinal axis of the ultrasonic probe 15 comprises a radially asymmetric cross section and the length of the ultrasonic probe 15 is chosen to be resonant in the torsional mode. In the torsional mode of vibration, a transducer transmits ultrasonic energy received from the ultrasonic energy source 99 to the ultrasonic probe 15, causing the ultrasonic probe 15 to vibrate torsionally. The ultrasonic energy source 99 produces the electrical energy that is used to produce a torsional vibration along the longitudinal axis of the ultrasonic probe 15. The torsional vibration is a torsional oscillation whereby equally spaced points along the longitudinal axis of the ultrasonic probe 15 including the probe tip 9 vibrate back and forth in a short arc about the longitudinal axis of the ultrasonic probe 15. A section proximal to each of a plurality of torsional nodes and a section distal to each of the plurality of torsional nodes are vibrated out of phase, with the proximal section vibrated in a clockwise direction and the distal section vibrated in a counterclockwise direction, or vice versa. The torsional vibration results in an ultrasonic energy transfer to the biological material with minimal loss of ultrasonic energy that could limit the effectiveness of the ultrasonic medical device 11. The torsional vibration produces a rotation and a counterrotation along the longitudinal axis of the ultrasonic probe 15 that creates the plurality of torsional nodes and a plurality of torsional anti-nodes along a portion of the longitudinal axis of the ultrasonic probe 15 resulting in cavitation along the portion of the longitudinal axis of the ultrasonic probe 15 comprising the radially asymmetric cross section in a medium surrounding the ultrasonic probe 15 that ablates the biological material. An apparatus and method for an ultrasonic medical device operating in a torsional mode is described in Assignee's co-pending patent application U.S. Ser. No. 10/774,985 and the entirety of this application is hereby incorporated herein by reference.

In another embodiment of the present invention, the ultrasonic probe 15 is vibrated in a torsional mode and a transverse mode. A transducer transmits ultrasonic energy from the ultrasonic energy source 99 to the ultrasonic probe 15, creating a torsional vibration of the ultrasonic probe 15. The torsional vibration induces a transverse vibration along an active area of the ultrasonic probe 15, creating a plurality of nodes and a plurality of anti-nodes along the active area that result in cavitation in a medium surrounding the ultrasonic probe 15. The active area of the ultrasonic probe 15 undergoes both the torsional vibration and the transverse vibration.

Depending upon physical properties (i.e., length, diameter, etc.) and material properties (i.e., yield strength, modulus, etc.) of the ultrasonic probe 15, the transverse vibration is excited by the torsional vibration. Coupling of the torsional mode of vibration and the transverse mode of vibration is possible because of common shear components for the elastic forces. The transverse vibration is induced when the frequency of the transducer is close to a transverse resonant frequency of the ultrasonic probe 15. The combination of the torsional mode of vibration and the transverse mode of vibration is possible because for each torsional mode of vibration, there are many close transverse modes of vibration. By applying tension on the ultrasonic probe 15, for example by bending the ultrasonic probe 15, the transverse vibration is tuned into coincidence with the torsional vibration. The bending causes a shift in frequency due to changes in tension. In the torsional mode of vibration and the transverse mode of vibration, the active area of the ultrasonic probe 15 is vibrated in a direction not parallel to the longitudinal axis of the ultrasonic probe 15 while equally spaced points along the longitudinal axis of the ultrasonic probe 15 in a proximal section vibrate back and forth in a short arc about the longitudinal axis of the ultrasonic probe 15. An apparatus and method for an ultrasonic medical device operating in a transverse mode and a torsional mode is described in Assignee's co-pending patent application U.S. Ser. No. 10/774,898 and the entirety of this application is hereby incorporated herein by reference.

The present invention provides an apparatus and a method of removing an occlusion 16 from a vasculature 44 of a body through the combination of ultrasonic energy from an ultrasonic probe and a balloon 41 that transmits the transverse ultrasonic vibrations in a probe from the ultrasonic energy to the occlusion 16. The present invention provides an apparatus and method of effectively removing the occlusion 16 that is simple, user friendly, effective, time efficient, reliable and cost effective.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. An ultrasonic medical device comprising:
an ultrasonic probe having a proximal end, a distal end and a longitudinal axis therebetween;
a catheter surrounding the ultrasonic probe, the catheter having a proximal end, a distal end and longitudinal axis therebetween;
an inflation lumen located along the longitudinal axis of the catheter; and
a balloon supported by the catheter, an inner surface of the balloon in communication with the inflation lumen.

2. The device of claim 1 wherein the ultrasonic probe extends from the proximal end of the catheter to the distal end of the catheter.

3. The device of claim 1 wherein the balloon engages the catheter at an at least one engagement position along the longitudinal axis of the catheter.

4. The device of claim 1 wherein the inflation lumen comprises an at least one inflation opening along a longitudinal axis of the inflation lumen.

5. The device of claim 1 wherein the inflation lumen is located inside of the catheter.

6. The device of claim 1 wherein the catheter comprises an at least one fenestration along the longitudinal axis of the catheter.

7. The device of claim 1 wherein a transverse ultrasonic vibration from the ultrasonic probe vibrates at least a portion of the balloon.

8. The device of claim 1 wherein the balloon concentrates a transverse ultrasonic energy from the ultrasonic probe.

9. The device of claim 1 wherein the balloon expands a treatment area of a biological material destroying effect of the ultrasonic probe.

10. The device of claim 1 wherein the balloon increases a radial span of a biological material destroying effect of the ultrasonic probe.

11. The device of claim 1 wherein at least a portion of an outer surface of the balloon engages a biological material.

12. The device of claim 1 wherein the balloon focuses a biological material destroying effect of the ultrasonic probe.

13. The device of claim 1 wherein the balloon adapts to a contour of a vasculature.

14. The device of claim 1 wherein the balloon is a non-compliant balloon.

15. The device of claim 1 wherein a second balloon is located along the longitudinal axis of the catheter.

16. An ultrasonic medical device comprising:
a catheter having a proximal end, a distal end and a longitudinal axis therebetween;
a balloon having an outer surface, an inner surface, a proximal end and a distal end, the balloon engaging the catheter along the longitudinal axis of the catheter;
an elongated ultrasonic probe having a proximal end, a distal end and a longitudinal axis therebetween, the elongated ultrasonic probe extending through at least a portion of the longitudinal axis of the catheter; and
an inflation lumen in communication with the balloon, the inflation lumen located along the longitudinal axis of the catheter.

17. The device of claim 16 wherein the balloon is supported by the catheter between the proximal end and the distal end of the balloon.

18. The device of claim 16 wherein the inflation lumen comprises an at least one inflation opening along a longitudinal axis of the inflation lumen.

19. The device of claim 16 wherein the catheter comprises an at least one fenestration along the longitudinal axis of the catheter.

20. The device of claim 16 wherein the catheter surround at least a portion of the elongated ultrasonic probe.

21. The device of claim 16 wherein a transverse ultrasonic vibration from the elongated ultrasonic probe vibrates at least a portion of the balloon.

22. The device of claim 16 wherein the balloon expands a treatment area of an occlusion destroying effect of the elongated ultrasonic probe.

23. The device of claim 16 wherein the balloon focuses an occlusion destroying effect of the elongated ultrasonic probe.

24. The device of claim 16 wherein the balloon is a non-compliant balloon.

25. The device of claim 16 wherein a second balloon is located along the longitudinal axis of the catheter.

26. A method of ablating a biological material in a vasculature of a body comprising:
inserting an ultrasonic probe in the vasculature;
moving the ultrasonic probe adjacent to the biological material;
advancing a catheter comprising a balloon on an outer surface of the catheter over a longitudinal axis of the ultrasonic probe until the balloon is adjacent to the biological material;
inflating the balloon so at least a portion of an outer surface of the balloon engages the biological material;
energizing the ultrasonic probe to produce a transverse ultrasonic vibration that vibrates at least a portion of the balloon to ablate the biological material; and
inflating the balloon to a larger diameter to engage the biological material and energizing the ultrasonic probe to ablate the biological material.

27. The method of claim 26 further comprising repeatedly inflating the balloon to a larger diameter to engage the biological material and repeatedly energizing the ultrasonic probe to ablate the biological material.

28. The method of claim 26 wherein the balloon is inflated by providing a medium through an inflation lumen located within the catheter.

29. The method of claim 28 wherein the medium engages the inner surface of the balloon through an at least one inflation opening along a longitudinal axis of an inflation lumen.

30. The method of claim 26 wherein vibrating at least a portion of the ultrasonic probe by an ultrasonic energy source produces the transverse ultrasonic vibration along at least a portion of the balloon.

31. The method of claim 26 wherein the transverse ultrasonic vibration of the ultrasonic probe provides a plurality of transverse nodes and transverse anti-nodes along a portion of the longitudinal axis of the ultrasonic probe.

32. The method of claim 31 wherein the transverse anti-nodes are points of a maximum transverse energy along a portion of the longitudinal axis of the ultrasonic probe.

33. The method of claim 31 wherein the transverse anti-nodes cause a cavitation in a medium in communication with the ultrasonic probe in a direction not parallel to the longitudinal axis of the ultrasonic probe.

34. The method of claim 31 wherein more than one of the plurality of transverse anti-nodes are in communication with the biological material.

35. The method of claim 26 wherein the balloon transmits the transverse ultrasonic vibration to the biological material.

36. The method of claim 26 wherein the balloon concentrates a transverse ultrasonic energy to the biological material.

37. The method of claim 26 balloon expands a treatment area of a biological material destroying effect of the ultrasonic probe.

38. The method of claim 26 wherein the balloon increases a radial span of a biological material destroying effect of the ultrasonic probe.

39. The method of claim 26 wherein the balloon can support the transverse ultrasonic vibration along at least a portion of the longitudinal axis of the ultrasonic probe to ablate the biological material.

40. A method of removing an occlusion in a vasculature of a body comprising:
inserting a biocompatible material member inside the vasculature;
advancing the biocompatible material member in the vasculature until a portion of the biocompatible material member is adjacent to the occlusion;
moving a flexible ultrasonic probe through the biocompatible material member until the flexible ultrasonic probe is adjacent to the occlusion; and
activating an ultrasonic energy source to produce a transverse ultrasonic vibration along at least a portion of a longitudinal axis of the flexible ultrasonic probe that vibrates at least a portion of the biocompatible material member to remove the occlusion.

41. The method of claim 40 further comprising providing the biocompatible material member that is selected from the group consisting of a catheter, a balloon, a sheath, a heat sink and similar devices.

42. The method of claim 40 further comprising providing the biocompatible material member that has a proximal end and a distal end.

43. The method of claim 40 wherein at least the portion of an outer surface of the biocompatible material member engages the occlusion.

44. The method of claim 40 wherein the biocompatible material member increases a radial span of an occlusion destroying effect of the flexible ultrasonic probe.

45. The method of claim 40 wherein the biocompatible material member concentrates a transverse ultrasonic energy from the flexible ultrasonic probe to the occlusion.

46. The method of claim 40 wherein the transverse ultrasonic vibration of the flexible ultrasonic probe provides a plurality of transverse nodes and transverse anti-nodes along the portion of the longitudinal axis of the flexible ultrasonic probe.

47. The method of claim 40 wherein the occlusion comprises a biological material.

48. The method of claim 40 wherein the flexible ultrasonic probe and the biocompatible material member are for a single use on a single patient.

49. The method of claim 40 wherein the flexible ultrasonic probe and the biocompatible material member are disposable.
